(19) United States
(12) Patent Application Publication
(10) Pub. No.: US 2018/0303167 A1
(43) Pub. Date: Oct. 25, 2018

(54) CONVECTION HEATING SYSTEM

(71) Applicant: Robert Basil, Suwanee, GA (US)

(72) Inventor: Robert Basil, Suwanee, GA (US)

(21) Appl. No.: 16/020,314

(22) Filed: Jun. 27, 2018

Related U.S. Application Data
(63) Continuation-in-part of application No. 15/418,558, filed on Jan. 27, 2017.
(60) Provisional application No. 62/292,586, filed on Feb. 8, 2016.

(51) Int. Cl. A24F 47/00; A61M 11/04; H05B 6/10; H05B 6/04; H05B 6/06; H05B 6/38; H05B 6/40; H05B 6/44

(52) U.S. Cl. CPC A24F 47/008; A61M 11/042; H05B 6/108; H05B 6/44; H05B 6/06; H05B 6/38; H05B 6/40; H05B 6/04

(57) ABSTRACT

Implementations of a convection heating system are provided. The convection heating system may be configured for use with an inhalation device (e.g., a vaporizer). The convection heating system is configured to vaporize (i.e., atomize) the active chemical(s) in a smokeable product using heated air so they can be inhaled by a user. A convection heating system may comprise an induction heating system having a thermally conductive porous fill material positioned to be indirectly, or directly, heated by a field coil(s) positioned thereabout. In some implementations, the thermally conductive porous fill material may be positioned to draw heat from a bowl of the induction heating system, the heat is transferred to air passing through the porous fill material. In another implementation, the thermally conductive porous fill material may be directly heated by the field coil(s) positioned thereabout, the heat is transferred to air passing through the porous fill material.

### CROSS REFERENCE TO RELATED APPLICATION

This is a continuation-in-part application claiming the benefit of U.S. patent application Ser. No. 15/418,558, filed on Jan. 27, 2017, which claims the benefit of U.S. Provisional Application Ser. No. 62/292,586, filed on Feb. 8, 2016, the entireties of both applications are incorporated herein by reference.

### TECHNICAL FIELD

This disclosure relates to implementations of an induction heating system and a convection heating system.

### BACKGROUND

Smoking is the practice of burning a substance and breathing in the resulting smoke so that any active chemical compounds contained within the smoke are absorbed into the bloodstream. Various plants, mostly dried leaves (e.g., tobacco, marijuana, etc.), are used around the world for smoking due to the various chemical compounds (e.g., nicotine, THC, etc.) they contain and the effects these chemicals have on the human body. The combustion of the plant material being smoked results in the release of not only the desired active chemical compound(s) (e.g., nicotine, THC, etc.) but also a variety of known carcinogenic compounds and other potentially harmful chemicals. Therefore, it would be desirable if there was a way to heat a substance (e.g., tobacco, marijuana, etc.) sufficiently to release the desired active chemical compound(s) without causing it to combust and release carcinogens and other potentially harmful chemicals.

### SUMMARY OF THE INVENTION

Implementations of a convection heating system are provided. In some implementations, the convection heating system may be configured for use with an inhalation device (e.g., a smoking pipe, water pipe, and/or vaporizer. In some implementations, the convection heating system may be configured to vaporize (i.e., atomize) the active chemical(s) (e.g., nicotine, THC, etc.) in a smokeable product (e.g., tobacco or medicinal herbs), without combustion, using heated air (i.e., convection). In this way, the aerosolized active chemical(s) may then be inhaled by a user.

In some implementations, a convection heating system may comprise an induction heating system having a thermally conductive porous fill material positioned to be indirectly, or directly, heated by a field coil(s) positioned thereabout.

In some implementations, the thermally conductive porous fill material of the convection heating system may be positioned to draw heat from a bowl of the induction heating system, which is transferred to air passing through the porous fill material. In this way, the active chemical(s) (e.g., nicotine) in a smokeable product (e.g., tobacco or medicinal herbs) resting on a screen positioned over the thermally conductive porous fill material may be vaporized (i.e., atomize) by the heated air without combustion. In some implementations, when a screen is not included as part of a convection heating system, the smokeable product may be rested directly on the thermally conductive porous fill material.

In some implementations, the thermally conductive porous fill material may be shaped to fit within the interior of the bowl and make contact therewith. In this way, when the bowl is heated by eddy currents and/or magnetic hysteresis resulting from current being passed through the field coil(s), the thermally conductive porous fill material draws heat from the bowl that is subsequently transferred to air passing therethrough.

In some implementations, the thermally conductive porous fill material may be a thermally conductive wool (e.g., aluminum wool). In some implementations, the thermally conductive porous fill material may be multiple strands of thermally conductive material (e.g., aluminum) that are configured (e.g., shaped) to facilitate the flow of air therethrough and positioned to make contact with the bowl. In some implementations, the thermally conductive porous fill material may be any thermally conductive material that is configured to facilitate the flow of air therethrough.

In another implementation, the thermally conductive porous fill material of the convection heating system may be positioned within a field coil(s) of the induction heating system and directly heated thereby, the heat is transferred to air passing through the porous fill material. In this way, the active chemical(s) (e.g., nicotine) in a smokeable product (e.g., tobacco or medicinal herbs) resting on a screen positioned over the thermally conductive porous fill material may be vaporized (i.e., atomize) by the heated air without combustion. In some implementations, when a screen is not included as part of a convection heating system, the smokeable product may be rested directly on the thermally conductive porous fill material.

In some implementations, the thermally conductive porous fill material may be wrapped in an electrically insulating heat resistant tape (e.g., a polyimide film such a Kapton®). In this way, the electrically insulating heat resistant tape may be used to hold the thermally conductive porous fill material in position within the field coil(s) of the induction heating system.

In some implementations, an induction heating system may comprise a bowl, a first current bearing wire (i.e., a field coil), a second current bearing wire (i.e., a field coil), a power source (e.g., one or more batteries), and a first switch.

In some implementations, the bowl may comprise a bottom, a cylindrical side wall extending upwardly therefrom defining a bowl interior (or chamber), and an upper rim. In some implementations, the chamber of the bowl may be configured to be packed with a smokeable product(s) (e.g., tobacco, medicinal herbs, etc.). In some implementations, the bowl may further comprise a hole (also known as a draft hole) in the bottom. In this way, the bowl may be configured for use with a vaporizer, a traditional water pipe, and/or smoking pipe.

In some implementations, the bowl may be composed of any magnetically permeable material (e.g., steel, ferrite, etc.). In some implementations, the bowl may have a coating (e.g., titanium nitride) thereon to minimize or prevent oxidation (e.g., rust).

In some implementations, a portion of the first current bearing wire and the second current bearing wire are wrapped about the bowl, forming a coil thereabout. In some implementations, the current bearing wires are wrapped about the side wall of the bowl, between the bottom and upper rim thereof. In this way, the bowl is heated by eddy currents and/or magnetic hysteresis when current is passed through the current bearing wires. In some implementations, the current bearing wires are wrapped in an interleaved configuration about the cylindrical side wall of the bowl.

In some implementations, the power source may be conductively connected to either the first current bearing wire or the second current bearing wire through the use of a first switch (e.g., a field-effect transistor). In this way, the flow of current from the power source is alternated between the first current bearing wire and the second current bearing wire. Alternating the flow of current between the current bearing wires changes the magnetic field.

In some implementations, a layer of insulating material (e.g., ceramic insulation tape) may be placed between the bowl and the portions of the current bearing wires wrapped thereabout. In this way, heat generated through induction may be better retained by the bowl.

In another example implementation, the current bearing wires may be wrapped in an adjacent configuration about the cylindrical side wall of the bowl. When the current bearing wires are in the adjacent configuration, the first current bearing wire may be positioned above the second current bearing wire relative to the bottom of the bowl.

In yet another example implementation, the induction heating system may comprise a single current bearing wire wrapped thereabout, a power source, a first switch (e.g., a field-effect transistor), and a second switch (e.g., a field-effect transistor). In some implementations, the power source may be conductively connected to the current bearing wire through both the first switch and the second switch. In some implementations, through the use of the first switch and the second switch, the direction of the current through the current bearing wire is alternated. In this way, the rapidly alternating magnetic field generated thereby heats the bowl.

In still yet another example implementation, the induction heating system may comprise a bowl having a first current bearing wire and a second current bearing wire wrapped thereabout in an interleaved configuration, a power source, a first switch (e.g., a field-effect transistor), and a second switch (e.g., a field-effect transistor). In some implementations, the power source may be conductively connected to the first current bearing wire and the second current bearing wire through the first switch and the second switch, respectively. In some implementations, through the use of the first switch and the second switch, the induction heating system may be configured so that the first current bearing wire and the second current bearing wire are energized for different lengths of time that may or may not overlap. In this way, a greater degree of control may be had over the temperature of the bowl.

### BRIEF DESCRIPTION OF THE DRAWINGS

FIGS. 1A and 1B illustrate an example induction heating system according to the principles of the present disclosure.

FIG. 1C illustrates an implementation of the induction heating system shown in FIGS. 1A and 1B that includes an on/off switch.

FIGS. 2A and 2B illustrate another example induction heating system according to the principles of the present disclosure.

FIGS. 3A and 3B illustrate yet another example induction heating system according to the principles of the present disclosure.

FIG. 4 illustrates an example bowl constructed in accordance with the present disclosure.

FIG. 5 illustrates another example bowl constructed in accordance with the present disclosure.

FIGS. 6A and 6B illustrate an example implementation of an induction heating system that has been configured for use with a vaporizer.

FIG. 7 illustrates still yet another example induction heating system according to the principles of the present disclosure.

FIG. 8 illustrates an example convection heating system according to the principles of the present disclosure.

FIG. 9 illustrates another example convection heating system according to the principles of the present disclosure.

### DETAILED DESCRIPTION

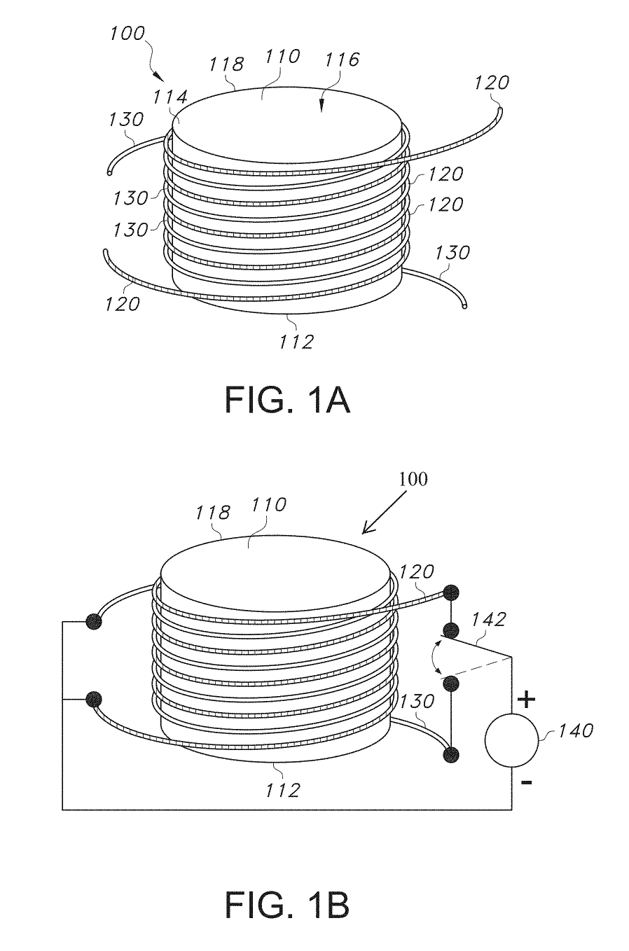

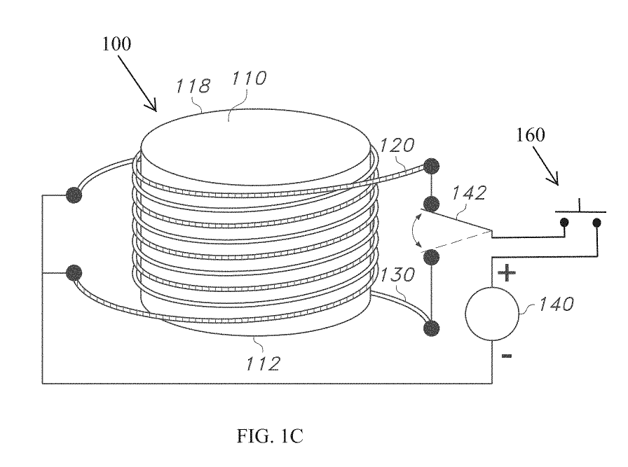

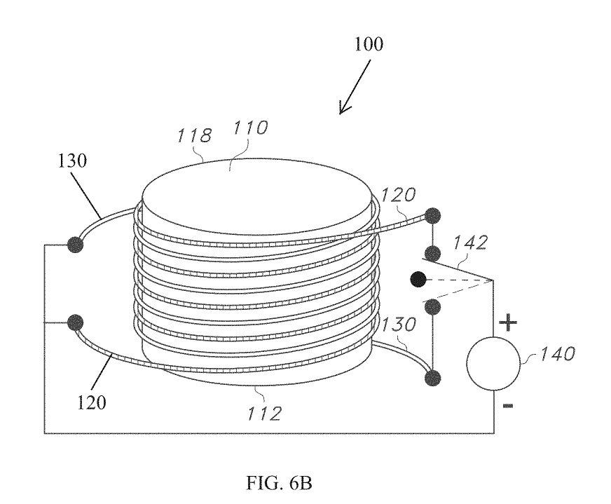

FIGS. 1A and 1B illustrate an example induction heating system 100 according to the principles of the present disclosure. In some implementations, the induction heating system 100 may be configured for use with a smoking pipe, water pipe, and/or vaporizer (see, e.g., FIG. 6A). In some implementations, the induction heating system 100 may be used to vaporize (i.e., atomize) the active chemical (e.g., nicotine) in a smokeable product (e.g., tobacco or medicinal herbs) without combustion. In this way, the aerosolized active chemical may then be inhaled by a user.

As shown in FIGS. 1A and 1B, in some implementations, an induction heating system 100 comprises a bowl 110, a first current bearing wire 120 (i.e., a field coil), a second current bearing wire 130 (i.e., a field coil), a power source 140 (e.g., one or more batteries), and a first switch 142.

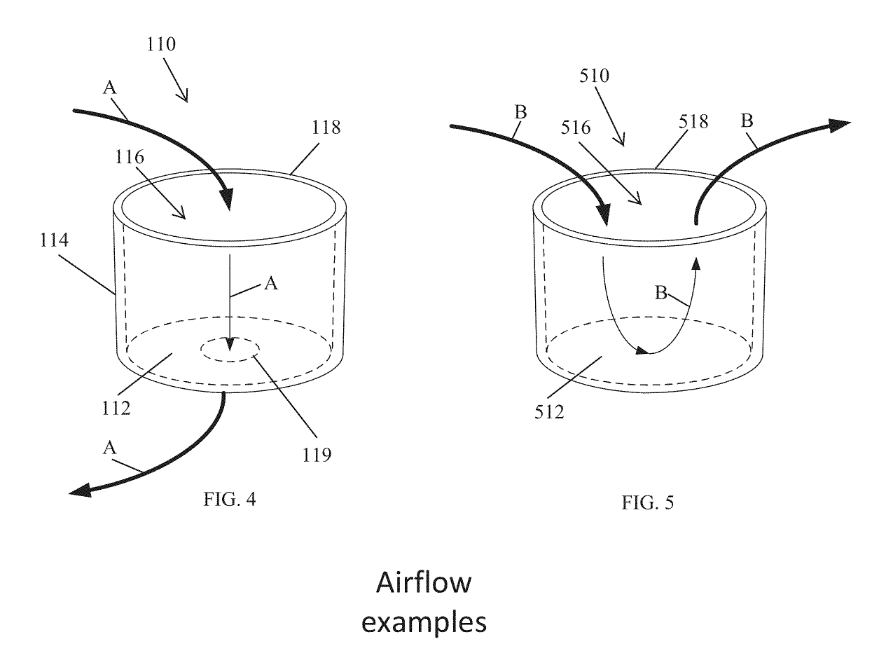

As shown in FIGS. 1 and 4, in some implementations, the bowl 110 may comprise a bottom 112, a cylindrical side wall 114 extending upwardly therefrom defining a bowl interior 116 (or chamber), and an upper rim 118. In some implementations, the chamber 116 of the bowl 110 may be configured to be packed with a smokeable product(s) (e.g., tobacco, medicinal herbs, etc.). In some implementations, the bowl 110 may further comprise a hole 119 (also known as a draft hole) in the bottom 112 (see, e.g., FIG. 4). In this way, the bowl 110 may be configured for use with a vaporizer, a traditional water pipe, and/or smoking pipe. In some implementations, the bowl 110 may be configured so that air A flows through the opening defined by the upper rim 118, into the interior 116 of the bowl 110, and out of the interior 116 through the hole 119 in the bottom 112 (see, e.g., FIG. 4). In this way, suction may be used to draw the vaporized active chemical(s) (e.g., nicotine) of a smokeable product into the lungs of a user. In some implementations, the bowl 110 may be any shape suitable for use as part of an induction heating system 100.

In some implementations, the bowl 110 may be configured to be secured to the smoke inlet tube of a vaporizer, water pipe and/or the shank of a smoking pipe. In some implementations, the bowl 110, 510 may be configured to be secured within an interior portion of a pipe and/or vaporizer.

In some implementations, the bowl 110 may be composed of steel. In some implementations, the bowl 110 may be composed of ferrite. In some implementations, the bowl 110 may be composed of any magnetically permeable material.

In some implementations, the bowl 110 may have a coating (e.g., titanium nitride) thereon to minimize or prevent oxidation (e.g., rust). In some implementations, the coating used to minimize or prevent oxidation of the bowl 110 may be any material, or combination of materials, that is resistant to high temperatures and/or is non-toxic to humans if inhaled.

As shown in FIG. 4, in some implementations, the hole 119 may be smaller in diameter than the bottom 112 of the bowl 110. In some implementations, the hole 119 may be circular (see, e.g., FIG. 4). In some implementations, the hole 119 may be any suitable shape.

As shown in FIG. 1A, in some implementations, a portion of the first current bearing wire 120 and the second current bearing wire 130 (collectively current bearing wires 120, 130) are wrapped about the bowl 110, forming a coil thereabout. In some implementations, the current bearing wires 120, 130 are wrapped about the side wall 114 of the bowl, between the bottom 112 and upper rim 118 thereof (see, e.g., FIG. 1A). In this way, the bowl 110 is heated by eddy currents and/or magnetic hysteresis when current is passed through the current bearing wires 120, 130. In some implementations, the current bearing wires 120, 130 are wrapped in an interleaved configuration about the cylindrical side wall 114 of the bowl 110 (see, e.g., FIG. 1A).

In some implementations, a layer of insulating material (e.g., ceramic insulation tape) may be placed between the bowl 110 and the portions of the current bearing wires 120, 130 wrapped thereabout. In this way, heat generated through induction may be better retained by the bowl 110. In some implementations, there may be no insulating material placed between the bowl 110 and the portions of the current bearing wires 120, 130 wrapped thereabout.

As shown in FIG. 1B, in some implementations, the power source 140 may be conductively connected to either the first current bearing wire 120 or the second current bearing wire 130 through the use of the first switch 142 (e.g., a field-effect transistor). In this way, the flow of current from the power source 140 is alternated between the first current bearing wire 120 and the second current bearing wire 130. Alternating the flow of current between the current bearing wires 120, 130 changes the magnetic field.

In some implementations, the switch 142 may be configured to include a third pole and thereby configured to create a delay between the first current bearing wire 120 and the second current bearing wire 130 being energized (see, e.g., FIG. 6B). In this way, there may be a period of time in which no heat is being generated by either current bearing wire 120, 130.

As shown in FIG. 1C, in some implementations, the induction heating system (e.g., FIG. 1B, element 100) may be configured to incorporate an on/off switch 160 (e.g., a tactile dome switch).

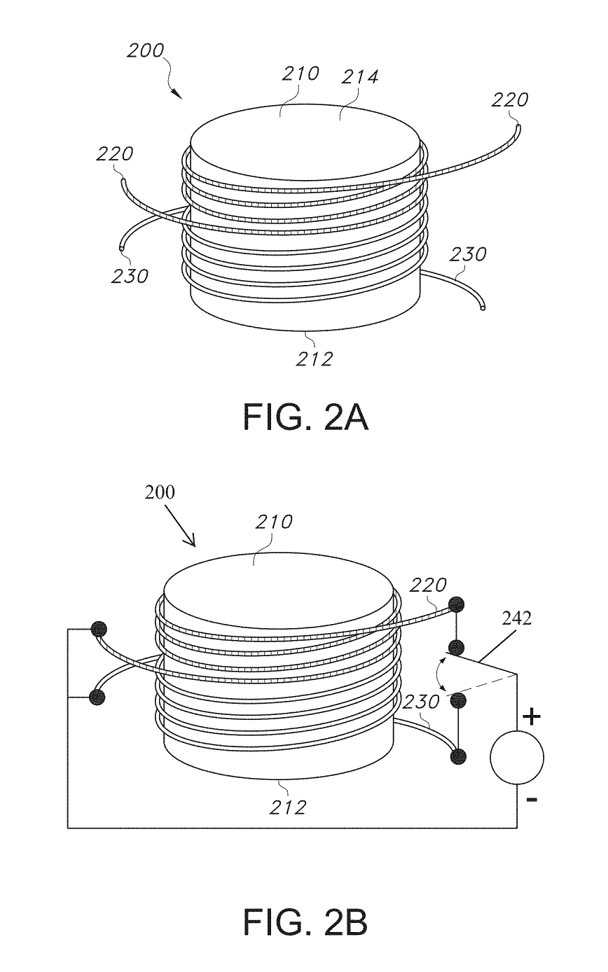

FIGS. 2A and 2B illustrate another example induction heating system 200. The induction heating system 200 is similar to the induction heating system 100 discussed above except the current bearing wires 220, 230 are wrapped in an adjacent configuration about the cylindrical side wall 214 of the bowl 210. In some implementations, when the current bearing wires 220, 230 are in the adjacent configuration, the first current bearing wire 220 is positioned above the second current bearing wire 230 relative to the bottom 212 of the bowl 210 (see, e.g., FIG. 2A). In some implementations, when the current bearing wires 220, 230 are in the adjacent configuration, the second current bearing wire 230 is positioned above the first current bearing wire 220 relative to the bottom 212 of the bowl 210 (not shown).

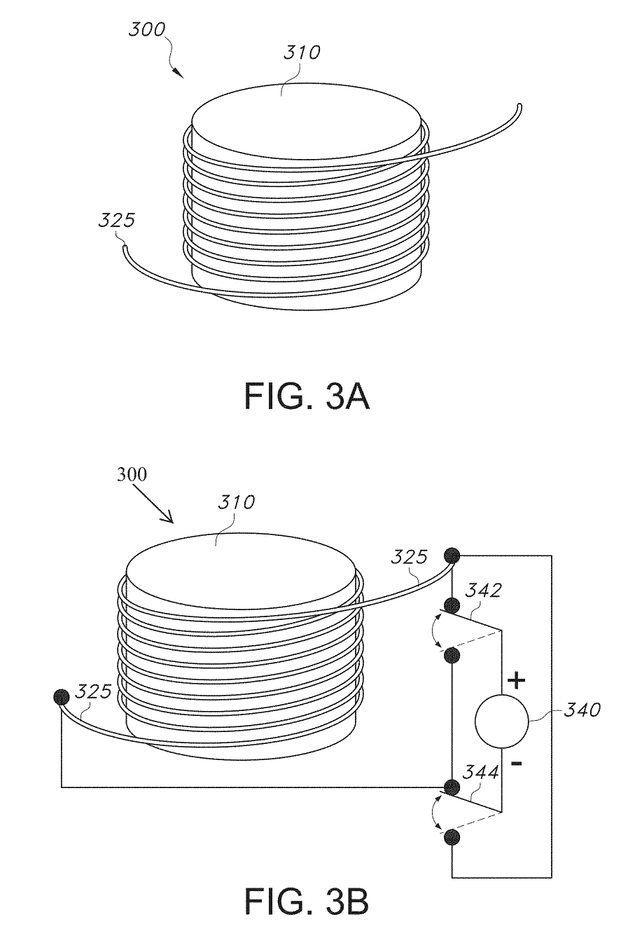

FIGS. 3A and 3B illustrate yet another example induction heating system 300. The induction heating system 300 is similar to the induction heating systems 100, 200 discussed above but instead comprises a bowl 310 having a single current bearing wire 325 (i.e., a field coil) wrapped thereabout, a power source 340, a first switch 342 (e.g., a field-effect transistor), and a second switch 344 (e.g., a field-effect transistor).

As shown in FIG. 3B, in some implementations, the power source 340 may be conductively connected to the current bearing wire 335 through both the first switch 342 and the second switch 344. In some implementations, through the use of the first switch 342 and the second switch 344, the direction of the flow of current through the current bearing wire 325 is alternated. In this way, the rapidly alternating magnetic field generated thereby heats the bowl 310.

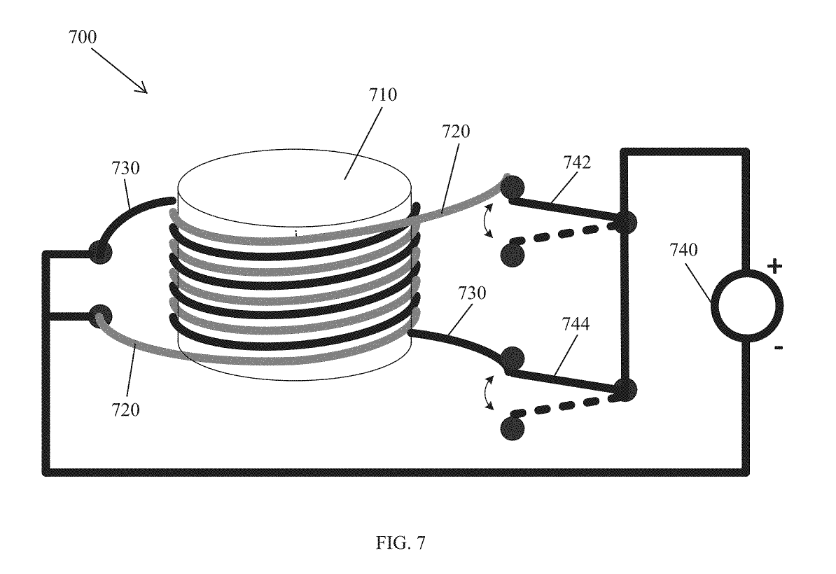

FIG. 7 illustrates still yet another example induction heating system 700. The induction heating system 700 is similar to the induction heating systems 100, 200, 300 discussed above but instead comprises a bowl 710 having a first current bearing wire 720 and a second current bearing wire 730 wrapped thereabout in an interleaved configuration, a power source 740, a first switch 742 (e.g., a field-effect transistor), and a second switch 744 (e.g., a field-effect transistor). In some implementations, the first switch 742 and the second switch 744 may be configured to independently control the flow of current from the power source 740 to the first current bearing wire 720 and the second current bearing wire 730.

As shown in FIG. 7, in some implementations, the power source 740 may be conductively connected to the first current bearing wire 720 and the second current bearing wire 730 through the first switch 742 and the second switch 744, respectively. In some implementations, through the use of the first switch 742 and the second switch 744, the first current bearing wire 720 and/or the second current bearing wire 730 may be used to heat the bowl 710.

In some implementations, through the use of the first switch 742 and the second switch 744, the induction heating system 700 may be configured so that the first current bearing wire 720 and the second current bearing wire 730 may be energized for the same or different lengths of time that may or may not overlap. In this way, a greater degree of control may be had over the heating of the bowl 710.

As shown in FIG. 7, in some implementations, the first switch 742 and the second switch 744 may be configured to conductively connect the first current bearing wire 720 and the second current bearing wire 730, respectively, to the power source 740 at the same time. In this way, the bowl 710 may be heated by eddy currents and/or magnetic hysteresis when current is passed through both current bearing wires 720, 730.

In some implementations, the first switch 742 and the second switch 744 may be configured to conductively connect the first current bearing wire 720 and the second current bearing wire 730, respectively, to the power source 740 at different times (i.e., only a single current bearing wire (720 or 730) may have current passing therethrough at any given time). In this way, the bowl 710 may be heated by eddy currents and/or magnetic hysteresis while current is passed through either the first current bearing wire 720 or the second current bearing wire 730.

In some implementations, the first switch 742 and the second switch 744 may be configured to conductively connect the first current bearing wire 720 and the second current bearing wire 730, respectively, to the power source 740 during overlapping intervals of time. In this way, the bowl 710 may be heated by eddy currents and/or magnetic hysteresis when current is passed through one or both current bearing wires 720, 730.

In some implementations, the first switch 742 and the second switch 744 may be configured to not conductively connect the first current bearing wire 720 and the second current bearing wire 730, respectively, to the power source 740 during overlapping intervals of time.

Although not shown in the drawings, it will be understood that suitable wiring connects the electronic components of the induction heating systems 100, 200, 300, 700 disclosed herein. It would be understood by one of ordinary skill in the art, that in some implementations, the induction heating system 100, 200, 300, 700 may be incorporated into a larger electrical circuit for use as part of a vaporizer, smoking pipe, and/or water pipe (see, e.g., FIGS. 6A and 6B).

As shown in FIG. 6A, in some implementation an induction heating system (e.g., induction heating system 100) may be configured for use as part of a vaporizer 600 comprising a bore 610, a control switch (e.g., element 160), and an electronic control board 670.

As shown in FIGS. 6A and 6B, in some implementations, the bore 610 may extend from the bottom 112 of the bowl 110 to an opening 605 in the exterior of the vaporizer 600. In this way, a user may draw the aerosolized chemical(s) generated by heating the smokeable product(s) resting in the chamber 116 of the bowl 110 through the bore 610 and into their mouth and/or lungs.

As shown in FIGS. 6A and 6B, in some implementations, the electronic control board 670 may be used in conjunction with the control switch (e.g., element 160) to control the operation of the first switch (e.g., 142, 242, 342, 742) and/or the second switch (e.g., 344, 744) of an induction heating system (e.g., 100, 200, 300, 700). In this way, the heat generated by the first field coil (e.g., 120, 220, 325, 720) and/or the second field coil (e.g., 130, 230, 730) may be regulated and/or the induction heating system may be turned on and/or off. In some implementations, the electronic control board 670 may be configured to position the first switch (e.g., 142, 242, 342, 742) and/or the second switch (e.g., 344, 744) into a third state in which neither the first current bearing wire (e.g., 120, 220, 325, 720) and/or the second current bearing wire (e.g., 130, 230, 720) (as appropriate) are being conductively completed thereby (see, e.g., FIG. 6B). In this way, the magnetic field(s) and thereby the amount of heat generated by the first field coil (e.g., 120, 220, 325, 720) and/or the second field coil (e.g., 130, 230, 730) may be regulated (e.g., increased, decreased, or stopped). One of ordinary skill in the art having the benefit of the present disclosure would know how to program/configure the electronic control board 670 to work as part of an induction heating system 100, 200, 300, 700.

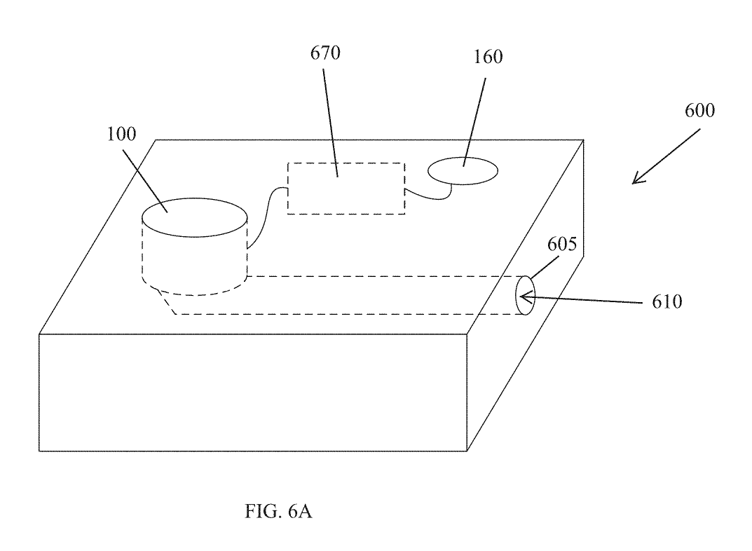

FIG. 5 illustrates another example implementation of the bowl 510 in accordance with the present disclosure. The bowl 510 is similar to the bowl 110 disclosed above except that there is no hole 119 through the bottom 512 (see, e.g., FIG. 5). In some implementations the bowl 510 may be configured so that air B flows into, and out of, the bowl interior 516 through the opening defined by the upper rim 518 thereof (see, e.g., FIG. 5).

In some implementations, the bowl 510 may be used in place of the bowl 110.

In some implementations, a top or lid may be used to cover the chamber 116, 516 of the bowl 110, 510. In this way, heat generated within the chamber 116, 516 may be trapped therein. In some implementations, a top or lid may not be used to cover the chamber 116, 516. In some implementations, the top or lid may be configured to secure about the upper rim 118, 518 of the bowl 110, 510.

In some implementations, the bowl may be a hollow cylinder without a top or a bottom that is configured to receive and retain a cartridge containing a liquid, or a secondary bowl, therein. In some implementations, the secondary bowl may be composed of a magnetically permeable material (e.g., steel, ferrite, etc.). In some implementations, the secondary bowl may be composed of ceramic. In some implementations, when the secondary bowl is resting within a hollow cylinder bowl, the secondary bowl may be heated by thermal conduction and/or through induction heating if the secondary bowl is composed of a magnetically permeable material.

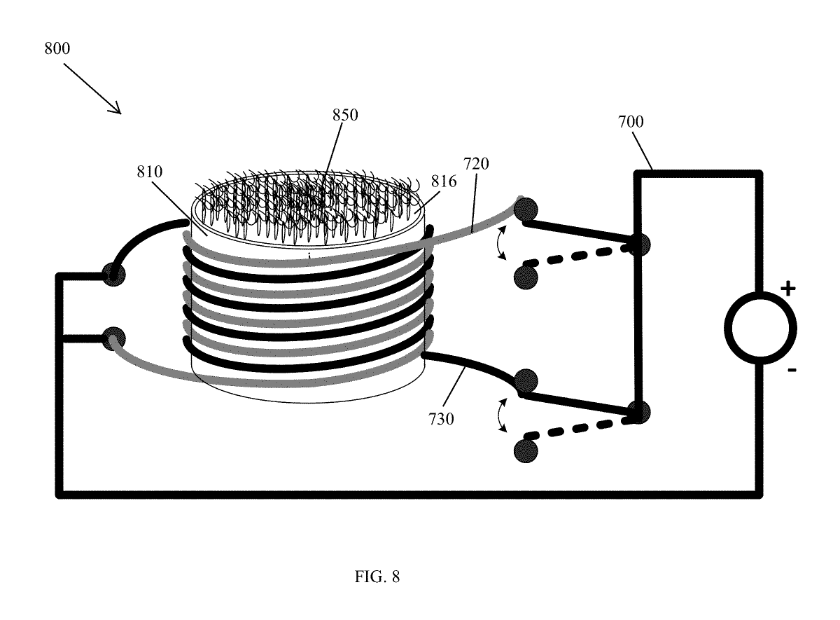

FIG. 8 illustrates an example convection heating system 800 according to the principles of the present disclosure. In some implementations, the convection heating system 800 may be configured for use with an inhalation device (e.g., the vaporizer shown in FIG. 6A). In some implementations, the convection heating system 800 may be configured to vaporize (i.e., atomize) the active chemical(s) (e.g., nicotine) in a smokable product (e.g., tobacco or medicinal herbs), without combustion, using heated air. In this way, the aerosolized active chemical(s) may then be inhaled by a user.

As shown in FIG. 8, in some implementations, a convection heating system 800 is similar to the induction heating systems 100, 200, 300, 700 discussed above, in particular the induction heating system 700 shown in FIG. 7, but further comprises a thermally conductive porous fill material 850 positioned within a bowl 810 thereof. In some implementations, the thermally conductive porous fill material 850 draws heat from the bowl 810 which is transferred to air passing through the porous fill material 850. In this way, the active chemical(s) (e.g., nicotine) in a smokeable product (e.g., tobacco or medicinal herbs) resting on a screen positioned over the thermally conductive porous fill material 850 may be vaporized (i.e., atomize) by the heated air without combustion. In some implementations, when a screen is not included as part of a convection heating system 800, the smokeable product may be rested directly on the thermally conductive porous fill material 850.

As shown in FIG. 8, in some implementations, the bowl 810 may be similar to the bowls 110, 210, 310, 70 discussed above, in particular the bowl 710 shown in FIG. 7, but the bowl 810 may be a tube (e.g., a hollow elongated cylinder) having an opening in a first end and a second end thereof.

As shown in FIG. 8, in some implementations, the thermally conductive porous fill material 850 may be shaped to fit within the interior of the bowl 810 and make contact therewith. In this way, when the bowl 810 is heated by eddy currents and/or magnetic hysteresis resulting from current being passed through the current bearing wires 720, 730, the thermally conductive porous fill material 850 draws heat from the bowl 810 that is subsequently transferred to air passing therethrough. In some implementations, the thermally conductive porous fill material 850 may be configured to fictionally engage with the interior 816 (or chamber) of the bowl 810, thereby preventing the thermally conductive porous fill material 850 from falling through the bowl 810. In some implementations, a screen may be secured to the bottom (or second end) of the bowl 810 and the thermally conductive porous fill material 850 rested thereon. In this way, air can flow through the screen and the thermally conductive porous fill material 850 positioned within the interior 816 of the bowl 810.

In some implementations, the thermally conductive porous fill material 850 may be aluminum wool (see, e.g., FIG. 8). In some implementations, the thermally conductive porous fill material 850 may be copper and/or silver wool. In some implementations, the thermally conductive porous fill material 850 may be multiple strands of thermally conductive material (e.g., aluminum) that are configured (e.g., shaped) to facilitate the flow of air therethrough and positioned to make contact with the bowl 810. In some implementations, the thermally conductive porous fill material 850 may be any thermally conductive material that is configured to facilitate the flow of air therethrough.

In some implementations, the thermally conductive porous fill material 850 may have a coating (e.g., titanium nitride) thereon to minimize or prevent oxidation (e.g., rust). In some implementations, the coating used to minimize or prevent oxidation of the thermally conductive porous fill material 850 may be any material, or combination of materials, that is resistant to high temperatures and/or is non-toxic to humans if inhaled.

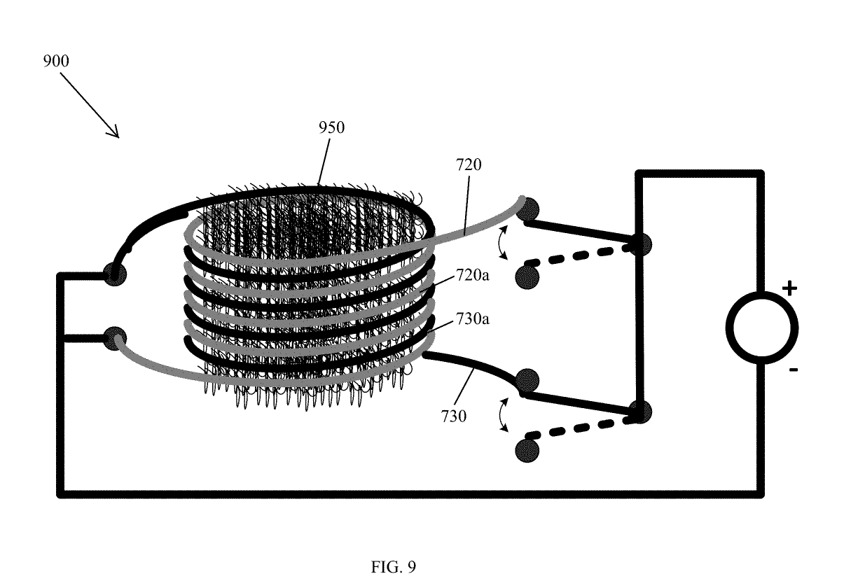

FIG. 9 illustrates another example convection heating system 900. The convection heating system 900 is similar to the convection heating system 800 discussed above but the thermally conductive porous fill material 950 is positioned within the field coils formed by the current bearing wires 720, 730, without the use of a bowl 710 or other device made of a magnetically permeable material. In this way, the thermally conductive porous fill material 950 may be directly heated by eddy currents and/or magnetic hysteresis resulting from current being passed through the current bearing wires 720, 730, heat is transferred from the thermally conductive porous fill material 950 to air passing therethrough. Thus, the active chemical(s) (e.g., nicotine) in a smokeable product (e.g., tobacco or medicinal herbs) resting on a screen positioned over the porous fill material 850 may be vaporized (i.e., atomize) by the heated air without combustion.

As shown in FIG. 9, in some implementations, at least a portion of the first current bearing wire 720 and the second current bearing wire 730 is formed into a first field coil 720a and a second field coil 730a, respectively. In some implementations, the first field coil 720a and the second field coil 730a are positioned so that the opening defined by the first field coil 720a is aligned (e.g., coaxially aligned) with the opening defined by the second field coil 730a. In this way, the thermally conductive porous fill material 950 may be positioned within the field coils 720a, 730a of the current bearing wires 720, 730 (see, e.g., FIG. 9). In some implementations, the first field coil 720a of the first current bearing wire 720 and the second field coil 730a of the second current bearing wire 730 are positioned in an interleaved configuration. In some implementations, the first field coil 720a of the first current bearing wire 720 and the second field coil 730a of the second current bearing wire 730 may be positioned in an adjacent configuration (see, e.g., the current bearing wires 220, 230 shown in FIGS. 2A and 2B). In some implementations, the induction heating system of the convection heating system 900 may only include a single current bearing wire having a single field coil (see, e.g., the current bearing wire 325 shown in FIGS. 3A and 3B).

In some implementations, the thermally conductive porous fill material 950 may be steel wool (see, e.g., FIG. 9). In some implementations, the thermally conductive porous fill material 950 may be any thermally conductive magnetically permeable material that is configured to facilitate the flow of air therethrough. In some implementations, the thermally conductive porous fill material 950 may be multiple strands of thermally conductive material (e.g., steel) that are configured (e.g., shaped) to facilitate the flow of air therethrough and positioned to make contact with the field coil(s) (e.g., field coils 720a and 730a).

In some implementations, the thermally conductive porous fill material 950 may be wrapped in an electrically insulating heat resistant tape (e.g., a polyimide film such a Kapton®). In this way, the electrically insulating heat resistant tape may be used to hold the thermally conductive porous fill material 950 in position within the field coils 720, 730 of a convection heating system 900.

In some implementations, a convection heating system 800, 900 provides a precise method of controlling the temperature of the air heated thereby, and the thermally conductive porous fill material 850, 950 increases the surface area, as compared to the single twisted coil of prior art solutions, used to heat air passing through an inhalation device.

Although not shown in the drawings, it will be understood that suitable wiring connects the electronic components of the convection heating systems 800, 900 disclosed herein. It would be understood by one of ordinary skill in the art, that in some implementations, the convection heating system 800, 900 may be incorporated into a larger electrical circuit for use as part of a vaporizer (e.g., the vaporizer 600 shown in FIGS. 6A and 6B), a smoking pipe, a water pipe, and/or other inhalation devices.

In some implementations, the one or more current bearing wires (e.g., current bearing wires 720, 730) of an induction heating system (e.g., the induction heating system 700 shown in FIG. 7) may be traces imbedded in an electrically insulating heat resistant tape (e.g., a polyimide film such a Kapton®). In this way, the electrically insulating heat resistant tape with embedded traces may be wrapped around a bowl (e.g., bowl 810), or around the thermally conductive porous fill material 950, of a conduction heating system 800, 900 and used to heat air passing through the thermally conductive porous fill material 850, 950.

In some implementations, a screen used as part of a convection heating system 800, 900 may be a mesh pipe screen, well known to one of ordinary skill in the art. In some implementations, a screen may be configured to rest on the upper rim 118 of a bowl (e.g., bowl 110). In some implementations, the screen may be configured to conform to the shape of the bowl interior 816 (or chamber).

In some implementations, a convection heating system 800, 900 may include a thermostat configured to regulate the temperature of air heated thereby. In this way, air passing through the thermally conductive porous fill material 850, 950 may be heated sufficiently to vaporize (i.e., atomize) the active chemical(s) (e.g., nicotine) in a smokable product (e.g., tobacco or medicinal herbs), without causing combustion. In some implementations, a thermostat may be embedded within the thermally conductive porous fill material 850, 950 of a convection heating system 800, 900. One of ordinary skill in the art, having the benefit for the present disclosure, would be able to select an appropriate thermostat and position it for use as part of a convection heating system 800, 900.

In some implementations, a fan may be used to move air through the thermally conductive porous fill material 850, 950 of a convection heating system 800, 900. In some implementations, a fan may be incorporated into a vaporizer (e.g., the vaporizer 600 shown in FIGS. 6A and 6B) that includes an implementation of a convection heating system 800, 900.

While the convection heating systems 800, 900 disclosed herein are shown in conjunction with the induction heating system 700 shown in FIG. 7, it is to be understood that one or more implementations of the convection heating system 800 900 disclosed herein may be used in conjunction with any suitable implementation of an induction heating system (e.g., the induction heating systems 100, 200, 300 shown in FIGS. 1A-1C, 2A-2B, and 3A-3B).

Reference throughout this specification to “an embodiment” or “implementation” or words of similar import means that a particular described feature, structure, or characteristic is included in at least one embodiment of the present invention. Thus, the phrase “in some implementations” or a phrase of similar import in various places throughout this specification does not necessarily refer to the same embodiment.

Many modifications and other embodiments of the inventions set forth herein will come to mind to one skilled in the art to which these inventions pertain having the benefit of the teachings presented in the foregoing descriptions and the associated drawings.

The described features, structures, or characteristics may be combined in any suitable manner in one or more embodiments. In the above description, numerous specific details are provided for a thorough understanding of embodiments of the invention. One skilled in the relevant art will recognize, however, that embodiments of the invention can be practiced without one or more of the specific details, or with other methods, components, materials, etc. In other instances, well-known structures, materials, or operations may not be shown or described in detail.

While operations are depicted in the drawings in a particular order, this should not be understood as requiring that such operations be performed in the particular order shown or in sequential order, or that all illustrated operations be performed, to achieve desirable results.

1. A convection heating system for use with an inhalation device, the convection heating system comprising:
a bowl, the bowl is a tube that includes an interior chamber defined by a side wall thereof, the tube includes an opening in a first end and a second end thereof;
a first current bearing wire and a second current bearing wire, wherein at least a portion of both the first current bearing wire and the second current bearing wire are wrapped about the side wall of the bowl;
a power source conductively connected to either the first current bearing wire or the second current bearing wire by a first switch; and
a thermally conductive porous fill material that is positioned within the interior chamber of the bowl;
wherein the first switch is configured to alternate the flow of current from the power source between the first current bearing wire and the second current bearing wire; and
wherein the thermally conductive porous fill material is positioned within the interior chamber of the bowl to make contact therewith.

2. The convection heating system of claim 1, further comprising a thermostat configured to regulate the temperature of air heated by the thermally conductive porous fill material.

3. The convection heating system of claim 1, wherein the first current bearing wire and the second current bearing wire are wrapped in an interleaved configuration about the side wall of the bowl.

4. The convection heating system of claim 3, wherein the bowl is composed of a magnetically permeable material.

5. The convection heating system of claim 4, wherein the bowl and the thermally conductive porous fill material include a titanium nitride coating thereon.

6. The convection heating system of claim 3, wherein a layer of insulating material is positioned between the bowl and the portions of the first current bearing wire and the second current bearing wire wrapped about the side wall of the bowl.

7. The convection heating system of claim 3, wherein the first switch is a field-effect transistor.

8. The convection heating system of claim 1, wherein the first current bearing wire and the second current bearing wire are wrapped in an adjacent configuration about the side wall of the bowl.

9. The convection heating system of claim 8, wherein the bowl is composed of a magnetically permeable material.

10. The convection heating system of claim 9, wherein the bowl and the thermally conductive porous fill material include a titanium nitride coating thereon.

11. The convection heating system of claim 8, wherein a layer of insulating material is positioned between the bowl and the portions of the first current bearing wire and the second current bearing wire wrapped about the side wall of the bowl.

12. The convection heating system of claim 8, wherein the first switch is a field-effect transistor.

13. A convection heating system for use with an inhalation device, the convection heating system comprising:
a bowl, the bowl is a tube that includes an interior chamber defined by a side wall thereof, the tube includes an opening in a first end and a second end thereof;
a first current bearing wire, wherein at least a portion of the first current bearing wire is wrapped about the side wall of the bowl;
a power source conductively connected to the first current bearing wire by a first switch and a second switch; and
a thermally conductive porous fill material that is positioned within the interior chamber of the bowl;
wherein the first switch and the second switch are configured to alternate the direction of the flow of current from the power source through the first current bearing wire; and
wherein the thermally conductive porous fill material is positioned within the interior chamber of the bowl to make contact therewith.

14. The convection heating system of claim 13, further comprising a thermostat configured to regulate the temperature of air heated by the thermally conductive porous fill material.

15. The convection heating system of claim 13, wherein the bowl is composed of a magnetically permeable material.

16. The convection heating system of claim 15, wherein the bowl and the thermally conductive porous fill material include a titanium nitride coating thereon.

17. The convection heating system of claim 13, wherein a layer of insulating material is positioned between the bowl and the portion of the first current bearing wire wrapped about the side wall of the bowl.

18. The convection heating system of claim 13, wherein the first switch is a field-effect transistor and the second switch is a field-effect transistor.

19. A convection heating system for use with an inhalation device, the convection heating system comprising:
a bowl, the bowl is a tube that includes an interior chamber defined by a side wall thereof, the tube includes an opening in a first end and a second end thereof;
a first current bearing wire and a second current bearing wire, wherein at least a portion of both the first current bearing wire and the second current bearing wire are wrapped about the side wall of the bowl;
a power source conductively connected to the first current bearing wire and the second current bearing wire by a first switch and a second switch, respectively; and
a thermally conductive porous fill material that is positioned within the interior chamber of the bowl;
wherein the first switch and the second switch are configured to independently control the flow of current from the power source through the first current bearing wire and the second current bearing wire; and
wherein the thermally conductive porous fill material is positioned within the interior chamber of the bowl to make contact therewith.

20. The convection heating system of claim 19, further comprising a thermostat configured to regulate the temperature of air heated by the thermally conductive porous fill material.

21. The convection heating system of claim 19, wherein the first current bearing wire and the second current bearing wire are wrapped in an interleaved configuration about the side wall of the bowl.

22. The convection heating system of claim 21, wherein the bowl is composed of a magnetically permeable material.

23. The convection heating system of claim 22, wherein the bowl and the thermally conductive porous fill material include a titanium nitride coating thereon.

24. The convection heating system of claim 21, wherein a layer of insulating material is positioned between the bowl and the portions of the first current bearing wire and the second current bearing wire wrapped about the side wall of the bowl.

25. The convection heating system of claim 21, wherein the first switch is a field-effect transistor and the second switch is a field-effect transistor.

26. A convection heating system for use with an inhalation device, the convection heating system comprising:
a first current bearing wire and a second current bearing wire, wherein at least a portion of the first current bearing wire is formed into a first field coil and at least a portion of the second current bearing wire is formed into a second field coil, the first field coil and the second field coil each define an opening, the first field coil and the second field coil are positioned so that the opening defined by the first field coil is aligned with the opening defined by the second field coil;
a power source conductively connected to either the first current bearing wire or the second current bearing wire by a first switch; and
a thermally conductive porous fill material that is positioned within the aligned openings of the first field coil and the second field coil;
wherein the first switch is configured to alternate the flow of current from the power source between the first current bearing wire and the second current bearing wire.

27. The convection heating system of claim 26, further comprising a thermostat configured to regulate the temperature of air heated by the thermally conductive porous fill material.

28. The convection heating system of claim 26, wherein the thermally conductive porous fill material is also a magnetically permeable material.

29. The convection heating system of claim 28, wherein the thermally conductive porous fill material is wrapped in an electrically insulating heat resistant tape.

30. The convection heating system of claim 26, wherein the first field coil of the first current bearing wire and the second field coil of the second current bearing wire are positioned in an interleaved configuration.

31. The convection heating system of claim 30, wherein the first switch is a field-effect transistor.

32. The convection heating system of claim 26, wherein the first field coil of the first current bearing wire and the second field coil of the second current bearing wire are positioned in an adjacent configuration.

33. The convection heating system of claim 32, wherein the first switch is a field-effect transistor.

34. A convection heating system for use with an inhalation device, the convection heating system comprising:
a first current bearing wire, wherein at least a portion of the first current bearing wire is formed into a field coil that defines an opening;
a power source conductively connected to the first current bearing wire by a first switch and a second switch; and
a thermally conductive porous fill material that is positioned within the opening defined by the field coil of the first current bearing wire;
wherein the first switch and the second switch are configured to alternate the direction of the flow of current from the power source through the first current bearing wire.

35. The convection heating system of claim 34, further comprising a thermostat configured to regulate the temperature of air heated by the thermally conductive porous fill material.

36. The convection heating system of claim 34, wherein the thermally conductive porous fill material is also a magnetically permeable material.

37. The convection heating system of claim 36, wherein the thermally conductive porous fill material is wrapped in an electrically insulating heat resistant tape.

38. The convection heating system of claim 34, wherein the first switch is a field-effect transistor and the second switch is a field-effect transistor.

39. A convection heating system for use with an inhalation device, the convection heating system comprising:
a first current bearing wire and a second current bearing wire, wherein at least a portion of the first current bearing wire is formed into a first field coil and at least a portion of the second current bearing wire is formed into a second field coil, the first field coil and the second field coil each define an opening, the first field coil and the second field coil are positioned so that the opening defined by the first field coil is aligned with the opening defined by the second field coil;
a power source conductively connected to the first current bearing wire and the second current bearing wire by a first switch and a second switch, respectively; and
a thermally conductive porous fill material that is positioned within the aligned openings of the first field coil and the second field coil;
wherein the first switch and the second switch are configured to independently control the flow of current from the power source through the first current bearing wire and the second current bearing wire.

40. The convection heating system of claim 39, further comprising a thermostat configured to regulate the temperature of air heated by the thermally conductive porous fill material.

41. The convection heating system of claim 39, wherein the thermally conductive porous fill material is also a magnetically permeable material.

42. The convection heating system of claim 41, wherein the thermally conductive porous fill material is wrapped in an electrically insulating heat resistant tape.

43. The convection heating system of claim 39, wherein the first field coil of the first current bearing wire and the second field coil of the second current bearing wire are positioned in an interleaved configuration.

44. The convection heating system of claim 43, wherein the first switch is a field-effect transistor and the second switch is a field-effect transistor.
